(19) United States
(12) Patent Application Publication
(10) Pub. No.: US 2022/0280798 A1
(43) Pub. Date: Sep. 8, 2022

(54) HEART MONITORING SYSTEM

(71) Applicant: Jason Burr, Red Lodge, MT (US)

(72) Inventor: Jason Burr, Red Lodge, MT (US)

(21) Appl. No.: 17/194,356

(22) Filed: Mar. 8, 2021

(51) Int. Cl. A61N 1/39; G08B 3/10; A61N 1/32; A61B 5/318

(52) U.S. Cl. CPC A61N 1/3904; G08B 3/10; A61N 1/321; A61B 5/318

(57) ABSTRACT

A heart monitoring system, including a heart defibrillation device, including a chest strap, a plurality of sensors disposed on at least a portion of the chest strap to monitor heart-related information, and a plurality of electrodes disposed on at least a portion of the chest strap to send at least one electric shock on each surface thereof in response to the plurality of sensors determining an emergency condition, and a heart monitoring device connected to the heart defibrillation device to indicate the emergency condition and emit an alarm sound in response to the plurality of sensors determining the emergency condition.

### BACKGROUND

### 1. Field

The present general inventive concept relates generally to a monitoring system, and particularly, to a heart monitoring system.

### 2. Description of the Related Art

Some people are prone to serious health risks while engaged in strenuous exercise. In particular, strenuous exercise can increase a person's heart rate which is a problem when not monitored properly. The increased heart rate can cause the person to have sharp pains in the chest that could prevent the person from reaching urgent medical attention without assistance.

A conventional automated external defibrillator (AED) is heavy and large in size. In other words, the conventional AED is inconvenient for users to utilize in real time to provide aid to themselves and/or others. As such, during a serious emergency, the person will be unable to receive life-saving treatment quickly.

Therefore, there is a need for a heart monitoring system that includes an AED as well as means to alert third parties.

### SUMMARY

The present general inventive concept provides a heart monitoring system.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a heart monitoring system, including a heart defibrillation device, including a chest strap, a plurality of sensors disposed on at least a portion of the chest strap to monitor heart-related information, and a plurality of electrodes disposed on at least a portion of the chest strap to send at least one electric shock on each surface thereof in response to the plurality of sensors determining an emergency condition, and a heart monitoring device connected to the heart defibrillation device to indicate the emergency condition and emit an alarm sound in response to the plurality of sensors determining the emergency condition.

The plurality of electrodes may refrain from sending the at least one electric shock in response to the plurality of sensors detecting a plurality of hearts in contact thereof.

Each of the plurality of sensors may compare readings of the heart-related information to each other to determine whether at least one defective sensor exists among the plurality of sensors, and to prevent the plurality of electrodes from activating in response to detecting the at least one defective sensor.

The heart monitoring system may further include an alert unit disposed on at least a portion of the chest strap to emit the alarm sound in response to the plurality of sensors determining the emergency condition.

The heart monitoring system may further include a communication unit disposed on at least a portion of the chest strap to send an alarm signal and a GPS signal to at least one third party in response to the plurality of sensors determining the emergency condition.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a perspective view of a heart monitoring system, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Heart Monitoring System 100

Heart Defibrillation Device 110

Chest Strap 111

Fastener 111a

Sensor 112

Electrode 113

Alert Unit 114

Communication Unit 115

Power Source 116

Charging Port 117

Heart Monitoring Device 120

Limb Strap 121

Display Unit 122

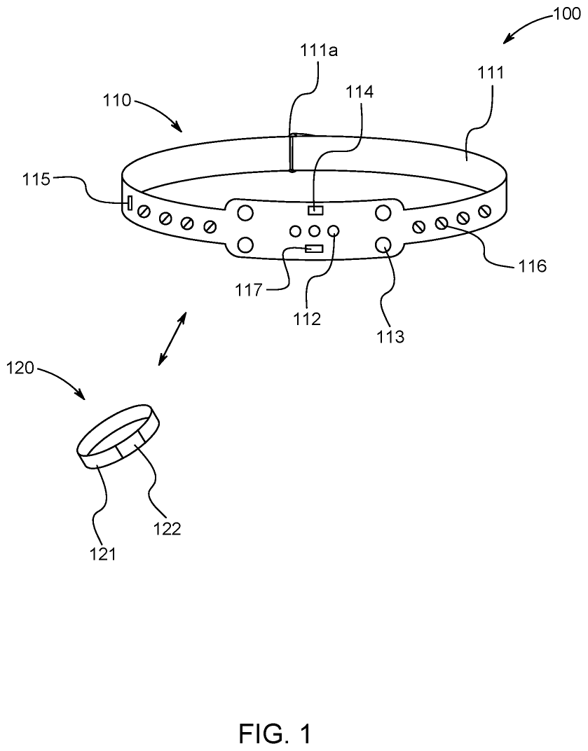

FIG. 1 illustrates a perspective view of a heart monitoring system 100, according to an exemplary embodiment of the present general inventive concept.

The heart monitoring system 100 may be constructed from at least one of metal, plastic, glass, cloth, fiber, and rubber, etc., but is not limited thereto. Also, the heart monitoring system 100 may be highly durable and waterproof.

The heart monitoring system 100 may include a heart defibrillation device 110 and a heart monitoring device 120, but is not limited thereto.

Referring to FIG. 1, the heart defibrillation device 110 is illustrated to fit around a chest of a user. However, the heart defibrillation device 110 may be sized for at least one limb (e.g., an arm, a leg), a neck, at least one finger, at least one toe, and/or any other size known to one of ordinary skill in the art, but is not limited thereto.

The heart defibrillation device 110 may include a chest strap 111, a plurality of sensors 112, a plurality of electrodes 113, an alert unit 114, a communication unit 115, a power source 116, and a charging port 117, but is not limited thereto.

The chest strap 111 may include a fastener 111a, but is not limited thereto.

The chest strap 111 may have a predetermined size, such as small, medium, and large. Alternatively, the chest strap 111 may be one-size-fits-all. In other words, the chest strap 111 may be flexible, such that the chest strap 111 may at least partially expand in response to being worn to accommodate the chest of the user, and/or any other part of a body of the user.

The fastener 111a may include a buckle, a clasp, a button, a snap, a clip, a zipper, hooks and loops, an adhesive (e.g., tape, glue), a magnet, and/or any combination thereof, but is not limited thereto.

The fastener 111a may be disposed on at least a portion of a first end of the chest strap 111. Moreover, the fastener 111a may facilitate adjustment of a size of the chest strap 111 during use. More specifically, the fastener 111a may allow a second end of the chest strap 111 to move toward and/or within the fastener 111a to decrease the size of the chest strap 111 during use. Alternatively, the fastener 111a may allow the second end of the chest strap 111 to move away from the fastener 111a to increase the size of the chest strap 111 during use.

Each of the plurality of sensors 112 may include a heart rate sensor, a blood pressure sensor, a vibration sensor, a temperature sensor, and a storage unit (e.g., a hard drive, a flash drive, etc.), but is not limited thereto.

Referring again to FIG. 1, although the plurality of sensors 112 are illustrated in plurality, the plurality of sensors 112 may be a single sensor 112. The plurality of sensors 112 may be disposed on at least a portion of the chest strap 111, such as a main portion of the chest strap 111 having a width greater than a remaining portion of the chest strap 111. Moreover, the plurality of sensors 112 may monitor heart-related information of the user, such as a heart rate level of the user, a blood pressure level of the user, a vibration level of a heart of the user (e.g., related to a heart arrythmia and/or palpitations), and/or a temperature level of the user. Additionally, the plurality of sensors 112 may compare the heart rate level to a predetermined normal heart rate level (e.g., 40 to 100 beats per minute (bpm)), compare the blood pressure level to a predetermined normal blood pressure level (e.g., systolic below 120 mm Hg, diastolic below 80 mm Hg), compare the vibration level to a predetermined normal vibration level, and/or compare the temperature level to a predetermined normal temperature level (e.g., 97.7 degrees F to 99.5 degrees F).

Referring again to FIG. 1, although the plurality of electrodes 113 are illustrated in plurality, the plurality of electrodes 113 may be a single electrode 113. The plurality of electrodes 113 may be disposed on at least a portion of the chest strap 111, such as the main portion of the chest strap 111. Furthermore, the plurality of electrodes 113 may be connected to each of the plurality of sensors 112. As such, the plurality of electrodes 113 may send at least one electric shock on a surface thereof in response to the plurality of sensors 112 determining an emergency condition (e.g., cardiac arrest, heart arrythmia, palpitations, and/or other abnormal heart conditions), based on the heart rate level exceeds the predetermined normal heart rate level, the blood pressure level exceeds the predetermined normal blood pressure level, the vibration level exceeds the predetermined normal vibration level, and/or the temperature level exceeds the predetermined normal temperature level. In other words, the plurality of electrodes 113 may discharge the at least one electric shock to a heart of the user to stabilize the heart of the user during the emergency condition.

However, the plurality of sensors 112 may monitor whether at least one other person is in contact with the user. As such, the plurality of electrodes 113 may refrain from sending the at least one electric shock in response to the at least one other person contacting the user to prevent injury to the at least one other user. For example, the plurality of sensors 112 may detect a plurality of hearts in contact thereof due to the plurality of heart rate levels from the user and the at least one other person contacting the user, a plurality of blood pressure levels from the user and the at least one other person contacting the user, a plurality of vibration levels from the user and the at least one other person contacting the user, and/or a plurality of temperature levels from the user and the at least one other person contacting the user.

Also, each of the plurality of sensors 112 may compare readings of the heart-related information to each other to determine whether an abnormality (e.g., at least one defective sensor) exists among the plurality of sensors 112. As such, an absence of the abnormality ensures proper operation of the plurality of sensors 112, and the plurality of sensors 112 may alert the user to a presence of the abnormality to prevent unnecessary injuries from the plurality of electrodes 113. As such, the plurality of sensors 112 may prevent the plurality of electrodes 113 from activating in response to detecting at least one defective sensor 112.

The alert unit 114 may include a display unit and a speaker, but is not limited thereto.

The alert unit 114 may be disposed on at least a portion of the chest strap 111, such as the main portion of the chest strap 111. The alert unit 114 may display a letter, a word, a phrase, a symbol, an image, and/or a picture thereon to indicate the emergency condition. Also, the alert unit 114 may emit an alarm sound and/or words identifying the emergency condition, such that the user and/or the at least one other person may be aware of the emergency condition.

The communication unit 115 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, global positioning system (GPS), Firewire, and Ethernet.

The communication unit 115 may be disposed on at least a portion of the chest strap 111. Moreover, the communication unit 115 may send an alarm signal to at least one third party (e.g., a family member, a doctor, a first responder, such as police, fire fighter, emergency medical technicians) in response to the plurality of sensors 112 determining the emergency condition. Furthermore, the communication unit 115 may send a global positioning system (GPS) signal to facilitate tracking thereof by the at least one third party. As such, the communication unit 115 may assist the user in receiving additional medical help.

The power source 116 may include a plurality of batteries and a solar cell, but is not limited thereto.

Referring again to FIG. 1, the power source 116 is illustrated to have a plurality of batteries. However, the power source 116 may be a single battery. The power source 116 may be disposed within at least a portion of the chest strap 111. The power source 116 may provide power to the plurality of sensors 112, the plurality of electrodes 113, the alert unit 114, and/or the communication unit 115.

The charging port 117 may include a universal serial bus (USB) port, but is not limited thereto.

The charging port 117 may be disposed on at least a portion of the chest strap 111, such as the main portion of the chest strap 111. The charging port 117 may connected to the power source 116 to charge the power source 116, such as the plurality of batteries in response to receiving an external power connection thereto (e.g., a power outlet). Moreover, the charging port 117 may also be used to connect an external device to download the heart-related information of the user and/or the emergency condition, including time of the event, as determined by the plurality of sensors 112. Additionally, the charging port 117 may allow the external device to download a time of the alarm sound from the alert unit 114 and the reason for the alarm sound (e.g., emergency condition). In other words, the plurality of sensors 112 may record important information that is downloadable for review by medical professionals.

Referring to FIG. 1, the heart monitoring device 120 is illustrated to fit around the at least one limb of the user. However, the heart monitoring device 120 may be sized for the neck, the at least one finger, the at least one toe, and/or any other size known to one of ordinary skill in the art, but is not limited thereto.

The heart monitoring device 120 may include a limb strap 121 and a display unit 122, but is not limited thereto.

The limb strap 121 may have a predetermined size, such as small, medium, and large. Alternatively, the limb strap 121 may be one-size-fits-all. In other words, the limb strap 121 may be flexible, such that the limb strap 121 may at least partially expand in response to being worn to accommodate the at least one limb of the user, and/or any other part of the body of the user.

The display unit 122 may include a plasma screen, an LCD screen, a light emitting diode (LED) screen, an organic LED (OLED) screen, a computer monitor, a hologram output unit, a sound outputting unit, or any other type of device that visually or aurally displays data.

The display unit 122 may further include a power source and a communication unit therein.

The display unit 122 may be disposed on at least a portion of the limb strap 121. The display unit 122 may display a letter, a word, a phrase, a symbol, an image, and/or a picture thereon to indicate the emergency condition. Similar to the alert unit 114, the display unit 122 may emit an alarm sound and/or words identifying the emergency condition, such that the user and/or the at least one other person may be aware of the emergency condition. The display unit 122 may connect to the communication unit 115 to receive the alarm signal from the communication unit 115, as well as other readings from the heart defibrillation device 110. In other words, the display unit 122 may allow the user and/or the at least one other person to be aware of the emergency condition where the user may have the heart defibrillation device 110 is hidden and/or out of view due to being on the chest of the user and under a shirt during exercise.

Therefore, the heart monitoring system 100 may prevent injury and/or death to the user during exercise and/or any other life-threatening emergency condition by providing a shock to the user when needed.

The present general inventive concept may include a heart monitoring system 100, including a heart defibrillation device 110, including a chest strap 111, a plurality of sensors 112 disposed on at least a portion of the chest strap 111 to monitor heart-related information, and a plurality of electrodes 113 disposed on at least a portion of the chest strap 111 to send at least one electric shock on each surface thereof in response to the plurality of sensors 112 determining an emergency condition, and a heart monitoring device 120 connected to the heart defibrillation device 110 to indicate the emergency condition and emit an alarm sound in response to the plurality of sensors 112 determining the emergency condition.

The plurality of electrodes 113 may refrain from sending the at least one electric shock in response to the plurality of sensors 112 detecting a plurality of hearts in contact thereof.

Each of the plurality of sensors 112 may compare readings of the heart-related information to each other to determine whether at least one defective sensor 112 exists among the plurality of sensors 112, and to prevent the plurality of electrodes 113 from activating in response to detecting the at least one defective sensor 112.

The heart monitoring system 100 may further include an alert unit 114 disposed on at least a portion of the chest strap 111 to emit the alarm sound in response to the plurality of sensors 112 determining the emergency condition.

The heart monitoring system 100 may further include a communication unit 115 disposed on at least a portion of the chest strap 111 to send an alarm signal and a GPS signal to at least one third party in response to the plurality of sensors 112 determining the emergency condition.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A heart monitoring system, comprising:
a heart defibrillation device, comprising:
a chest strap,
a plurality of sensors disposed on at least a first portion of the chest strap to monitor heart-related information, and
a plurality of electrodes disposed on at least a second portion of the chest strap to send at least one electric shock on each surface thereof in response to the plurality of sensors determining an emergency condition and refrain from sending the at least one electric shock in response to the plurality of sensors detecting a plurality of temperature levels; and
a heart monitoring device connected to the heart defibrillation device to indicate the emergency condition and emit a first alarm sound in response to the plurality of sensors determining the emergency condition.

2. The heart monitoring system of claim 1, wherein the plurality of electrodes refrain from sending the at least one electric shock in response to the plurality of sensors configured to detect a plurality of hearts in contact thereof.

3. The heart monitoring system of claim 1, wherein each of the plurality of sensors compares readings of the heart-related information to each other to determine whether at least one defective sensor exists among the plurality of sensors, and to prevent the plurality of electrodes from activating in response to detecting the at least one defective sensor.

4. The heart monitoring system of claim 1, further comprising:
an alert unit disposed on at least a portion of the chest strap to emit a second alarm sound in response to the plurality of sensors determining the emergency condition.

5. The heart monitoring system of claim 1, further comprising:
a communication unit disposed on at least a portion of the chest strap to send an alarm signal and a GPS signal to at least one third party in response to the plurality of sensors determining the emergency condition.

6. A heart monitoring system, comprising:
a heart defibrillation device, comprising:
a chest strap,
a plurality of sensors disposed on at least a first portion of the chest strap to monitor heart-related information, and
a plurality of electrodes disposed on at least a second portion of the chest strap to send at least one electric shock on each surface thereof in response to the plurality of sensors determining an emergency condition and refrain from sending the at least one electric shock in response to the plurality of sensors detecting an abnormality of at least one of the plurality of sensors; and
a heart monitoring device connected to the heart defibrillation device to indicate the emergency condition and emit a first alarm sound in response to the plurality of sensors determining the emergency condition.
